(19) United States
(12) Patent Application Publication
(10) Pub. No.: US 2012/0330556 A1
(43) Pub. Date: Dec. 27, 2012

(54) Analyte Testing System with Docking Station for Data Management

(75) Inventors: Gad Shaanan, La Jolla, CA (US); Marc Goldman, San Diego, CA (US)

(73) Assignee: GADLIGHT, INC., La Jolla, CA (US)

(21) Appl. No.: 13/308,024

(22) Filed: Nov. 30, 2011

Related U.S. Application Data
(63) Continuation of application No. 13/187,360, filed on Jul. 20, 2011.
Application No. 13/187,360 is a continuation-in-part of application No. 13/165,621, filed on Jun. 21, 2011.

(51) Int. Cl. G06F 19/00; G01N 33/02; G01N 33/483; G01N 33/15

(52) U.S. Cl. 702/19

(57) ABSTRACT

An analyte testing system having an analyte testing device and a docking station is described. The analyte testing device includes a plurality of lancets, a plurality of analyte sensors, electronics for deriving test data from the analyte sensors, a visual display that displays test data, and a data recording facility that records non-test data. Examples of non-test data include text diary information, audio diary information, food eaten, minutes exercised, medication taken, and estimated calories burned. The docking station has a power interface and data interface that provide power and data connectivity to the testing device. The docking station also has a storage facility configured to automatically store the test data and non-test data.

This application is a continuation of U.S. patent application Ser. No. 13/187,360 filed Jul. 20, 2011, which is a continuation-in-part of U.S. patent application Ser. No. 13/165,621 filed Jun. 21, 2011, both of which are incorporated herein by reference in their entirety.

### FIELD OF THE INVENTION

The field of the invention is analyte testing systems.

### BACKGROUND

Analyte testing systems play a critical roll in modern diagnosis and management of health-related issues. For example, a sample of human blood, urine, and/or saliva can be tested for glucose, fructosamine, hematocrit, hemoglobin blood oxygen saturation, lactates, iron, pH, cholesterol, liver enzymes (AST, ALT, alkaline phosphatase/GGT, LDH, bilirubin, etc), hormones, and other chemicals.

Numerous analyte testing systems comprising handheld analyte meters and docking stations are known in the art. U.S. Pat. No. 6,849,237 to Housefield, for example, discloses a docking station and a glucose meter for diabetic patients. The docking station provides electrical power to the meter and charges a rechargeable battery in the meter. The docking station also has a data interface and a storage medium for downloading data from the meter. In addition, the docking station can transmit data to a personal computer.

Other examples of known analyte testing systems having an analyte meter and docking station include: U.S. Pat. No. 7,883,015, U.S. 2002/0170823, U.S. 2004/0172284, U.S. 2006/0229502, U.S. 2006/0245131, U.S. 2008/0217407, WO2002/094092, WO2006/122741, EP1717924, CA2544953, and CA2599573.

WO2011060923 to Reinke discloses a handheld glucose meter that automatically backs up data to a portable remote device (e.g., cellular phone) via wireless communication. Data can include test data (e.g., glucose levels) and “non-test data” such as physical activities data (e.g., minutes exercised, calories burned), medication data (e.g., dosage amount, time of dosage), health status data (e.g., energy level, stress level, premenstrual, illness symptoms), and food data (e.g., food consumed, serving size, time). The portable device also has data management software for storing, analyzing, and monitoring the data.

It has yet to be appreciated that a docking station (a device that docks a portable device, but does not have a general purpose operating system) can store and manage test data and non-test data. For example, a docking station can include a storage medium, processor, and executable code configured to track supplies, reorder supplies, track dosages administered, recommend a dosage amount, alert a user or health care professional, correlate test data with non-test data, and/or transmit data to a health care server, all without having a general purpose operating system.

Thus, there is still a need for an analyte testing system that includes a hand-held analyte testing device, and a docking station that can store and manage test data and non-test data.

### SUMMARY OF THE INVENTION

The inventive subject matter provides apparatus, systems, and methods in which an analyte testing system comprises a hand-held analyte testing device and a docking station. The analyte testing device houses: (i) a plurality of lancets, (ii) a plurality of analyte sensors usable in conjunction with the lancets, (iii) electronics for deriving test data from the analyte sensors, (iv) a visual display that displays the test data, and (v) a data recording facility that records non-test data. The handheld device is used to prick a body member for drawing a blood sample, and test the blood sample for analytes such as glucose. The docking station has a power interface for providing power to the analyte testing device. The docking station also has a data interface and a storage facility that stores the test data and the non-test data.

In one aspect of preferred embodiments, the testing device and/or the docking station has a communication facility configured to transmit and receive with an external storage device (e.g., a medical provider server, home computer, local area network). The communication facility can be wired or wireless. In preferred embodiments, the communication facility uses at least two wireless protocols. In other preferred embodiments, the communication facility transmits data a cell phone network. Contemplated data include test data, information derived from test data, and non-test data.

In some aspects of preferred embodiments, the power interface is an inductive charging pad and the data interface is a wired connection, such as a USB port. The docking station can further include a second data interface, either wired or wireless, for exchanging data with an external device (e.g., home computer, laptop, smart phone, insulin pen, health test device).

The data collected by the analyte testing device electronics can include test data (i.e., analyte testing results), information derived from the test data (e.g., reports, graphs, analytics, trends), and even non-test data, such as: diary information recorded as text and/or audio; supplies used; supplies ordered; food eaten, minutes exercised and estimated calories burned; amount of medication taken, time medication was taken, supplies available in a user's personal inventory, and supply ordering history.

In some preferred embodiments, at least one of the docking station and testing device includes a panic button configured to alert a third party of a user's identity and condition. The docking station also preferably has a backup rechargeable battery that can replace a rechargeable battery of the handheld analyte testing device.

In other aspects of preferred embodiments, the testing device display is an LCD touch screen that indicates (i) whether the analyte testing device is charging and (ii) whether data is being transferred between the analyte testing device and the docking station. It is also contemplated that the docking station can include a display for indicating a charge status and data exchange status. The display preferably includes a graph showing a maximum threshold line, minimum threshold line, and a test data line. The max/min threshold lines indicate help the user to determine whether analyte test results are within acceptable limits.

In some aspects of preferred embodiments, the recording facility is a microphone and/or keypad, which can be used to record diary entries.

In yet other preferred embodiments, at least one of the testing device and the docking station has a processor programmed to test the adequacy of a communication link between the testing device and the docking station.

The docking station preferably has a processor and executable code that is configured to automatically (i) back-up the test data and the non-test data, (ii) track supply usage, and (iii) re-orders supplies.

In some aspects of preferred embodiments, the plurality of lancets are contained in a lancet cartridge and the plurality of analyte sensors are contained in an analyte sensor cartridge. It is also contemplated that the analyte sensor cartridge can be separate from the lancet cartridge.

In yet other aspects of preferred embodiments, the storage facility in the docking station is removable. The storage facility can also be configured to store data in a generic file format (e.g., pdf, doc, xml, eps, html, jpeg, rtf, and txt). Preferably, the storage facility is password protected and stores the test data and non-test data using encryption.

In other aspects of preferred embodiments, the analyte testing system also includes an insulin pen for administering medication. The pen preferably has a wireless communication facility configured to exchange data with the testing device.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1a and 1b are perspective views of one embodiment of an analyte testing system.

FIG. 2 is a perspective view of one embodiment of a handheld analyte testing device.

FIG. 3 is a perspective view of a back side of the handheld analyte testing device of FIG. 2.

FIG. 4 is a perspective view of a lancet cartridge.

FIG. 5 is a perspective view of an analyte sensor cartridge.

FIG. 6 is a perspective view of a docking station communicatively coupled with two external devices.

FIG. 7 is a perspective view of a handheld analyte testing device communicatively coupled with two external devices.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

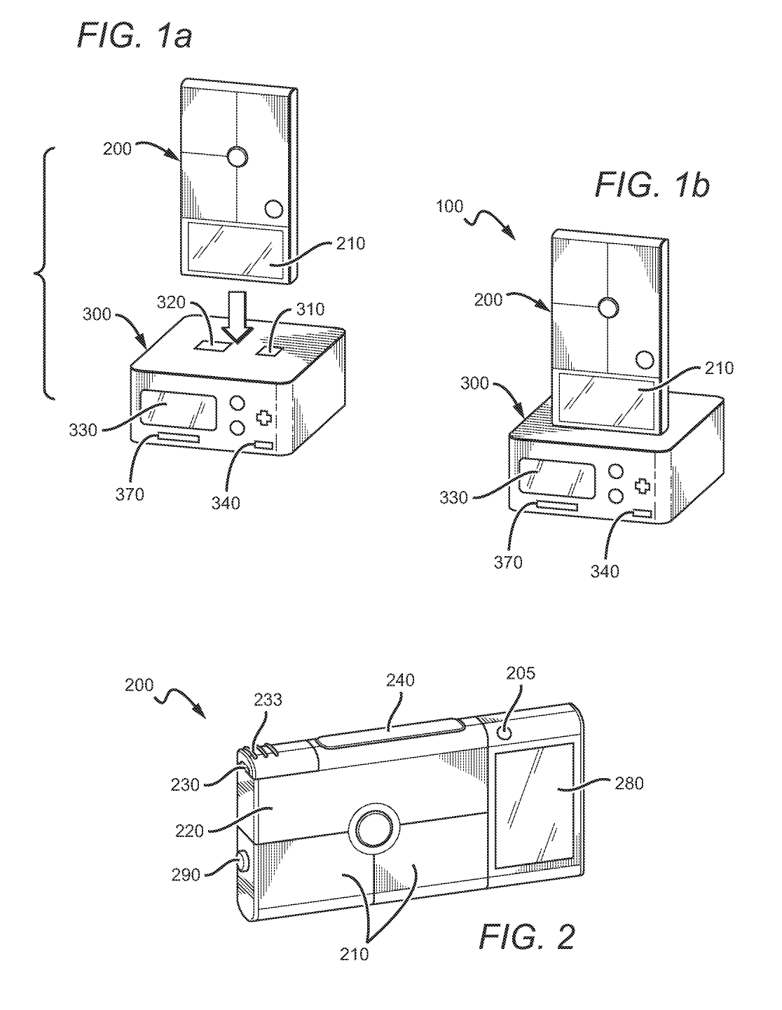

Figures la and lb show an analyte testing system 100 comprising a handheld analyte testing device 200 and a docking station 300. Device 200 couples with station 300 via power interface 310 and data interface 320, as shown in FIG. 1b. Docking station 300 can optionally be configured with a cradle or recessed portion for receiving device 200 in a secure manner. FIG. 1a shows device 200 disconnected from station 300. Power interface 310 is an inductive pad configured to provide electrical power to a rechargeable battery within device 200. An inductive pad advantageously allows device 200 to charge without physically mating with the docking station. In alternative embodiments, power interface 310 could comprise an electrical connector configured to mate with a connector on device 200. Data interface 320 is physical connector configured to mate with a connector on device 200 and provides data connectivity between storage mediums in device 200 and docking station 300 (not shown). However, it is also contemplated that data interface 320 could comprise a wireless transmitter communicatively coupled with a wireless transmitter of device 200.

Interfaces 310 and 320 can be two separate and distinct interfaces, or alternatively, can be integrated into one interface. Power interfaces and data interfaces are well known. In one embodiment, data interface 320 comprises a USB port and power interface 310 comprises an inductive current loop. Interfaces 310 and 320 can utilize an industry standard or proprietary technology. In some preferred embodiments, data interface 320 is a wireless transceiver configured to communicate with a wireless transceiver of device 200 using any number of wireless communication protocols and technologies (e.g., Bluetooth, wifi, cellular network, 802.11). The term wifi is used here generically to refer to a wireless local area network, rather than in a trademark sense to refer to Wi-Fi™. In such embodiments, station 300 preferably uses at least two alternative wireless communication protocols so that a secondary communication link is available in case the primary communication link fails. Including multiple protocols also advantageously increases compatibility with other devices.

Docking station 300 has a removable storage medium 370 coupled with an internal processor/electronics (not shown), and is configured to automatically backup analyte testing results data from device 200. The internal electronics are also preferably configured to analyze test results and identify trends. In addition to test data, it is further contemplated that other data can be stored and analyzed on station 300 and/or device 200. Such data can include time-stamped diary entries, either in text or audio format. For example, a user can record verbal comments on his or her physical health (e.g., severity and frequency of symptoms). Other data can further include: medication taken (amount, time), supplies used, supply order history, supplies remaining in the user's personal inventory, exercise (minutes, type), estimated calories burned, dietary intake information (protein, sugar, fat, sodium, etc) or any other information relevant and helpful for monitoring analytes and health issues. Contemplated supplies include, but are not limited to, lancets, test strips, and medication.

A removable storage medium 370 advantageously allows a user to take the storage facility to a health care provider for sharing the health data. The data is preferably password protected and/or encrypted in order to maintain the user's privacy. The data is preferably stored in a common or standard format (e.g., pdf, doc, xml, eps, html, jpeg, rtf, and txt) so that a doctor can view the data without the need for custom software.

Docking station 300 has an LCD touch screen display 330 for displaying and inputting information. For example, display 330 can show test results, history and trending of test results, supplies used, supplies remaining, or any other data helpful for monitoring analytes and health. In preferred embodiments, display 330 shows the number of lancet and test strip cartridges remaining in the user's personal inventory (e.g., closet). Display 330 can further be used for video conference communication with a health care professional or for displaying instructional videos on how to operate system 100. Display 330 can additionally serve as an input device for recording audio diary entries, dietary or exercise information, or any other data useful for monitoring analytes. However, it is also contemplated that an input device other than display 330 can be included in station 300 (e.g., buttons, key paid, microphone for voice-recognition commands).

Display 330 can further display an indication of whether device 200 is properly connected via power interface 310 and data interface 320. In addition, display 330 can show whether power interface 310 is rechargeable charging a battery of device 200, estimated time to complete a full charge, and whether data is currently being exchanged via in interface 320. The indicators can optionally comprise several LED lights having different colors and/or different blinking patterns.

Docking station 300 can include additional data interfaces and can be configured to function as a hub for multiple handheld health monitoring devices. In this manner, docking station 300 can act as a central point for gather a user's health data, analyzing the data, and transmitting the data to a health care provider. Station 300 can also include a calibration mechanism for testing accuracy of device 200. In addition, station 300 can include electronics for testing the adequacy and operability of the testing device's communication facility.

FIG. 2 shows handheld analyte testing device 200. Device 200 is a lancing device integrated with an analyte meter. The housing of device 200 has a first compartment 210 and a second compartment 220, for storing a lancet cartridge 215 (see FIG. 4) and a analyte sensor cartridge 225 (see FIG. 5), respectively. Device 200 houses various electrical components (memory, processor, executable code, etc) configured to convert electrical signals from analyte test strip 226 (see FIG. 5) into a test result reading. Device 200 also has a data recording facility 205 for recording data, and a display 280 for displaying data.

Data recording facility 205 and related electronics are used to store voice recordings of diary information as previously described. The electronics can also include a speaker for communicating data to a user and for prompting a user to use the device according to a pre-selected time and/or pre-selected time interval. In addition, the electronics can be equipped with an accelerometer or pedometer for measuring and calculating distance traveled and calories burned. In other aspects of preferred embodiments, the electronics of device 200 preferably includes a processor programmed to correlate individual instances of data with time stamps. For example, test result data and diary entries can be time stamped and correlated. In addition, the processor can be programmed to make an evaluation of the data, and send a notification to different recipients as a function of the evaluation. In some preferred embodiments, the processor is used to create static reports and saved in a generic file format onto removable storage medium 370. Yet still, the processor can be programmed to keep track of inventory of lancets and test strips, automatically re-order supplies, and automatically backup data to storage medium 370.

Device 200 has an actuator 240 configured to (i) cock a lancing apparatus within device 200 (not shown), (ii) expose a test strip for use, and (iii) advance the lancet cartridge. The test strip is exposed via slot 230. An ejection mechanism 233 allows for ejection and disposal of the test strip after testing, without the need for directly touching the test strip.

Device 200 has a LCD touch screen display 280, which can be used in a similar fashion to display 330 of station 300. For example, display 280 can be used to input diary information using a touch screen keypad. It is also contemplated that device 200 can include a hard keypad. In preferred embodiments, display 280 is used to display a graph that shows actual test data in relation to a maximum and minimum threshold line. The electronics of device 200 can be programmed to notify the user or a medical care provider via a cellular network when test data exceeds the max/min thresholds.

Device 200 also has a panic button 290 that is configured to communicate the user's identity and health status to a third party. For example, the panic button can be used to contact an emergency service, identify the patient's name, current location, and health status. It is further contemplated that panic button 290 can be configured to contact different persons (e.g., relative, home nurse, doctor, police) and convey different levels of urgency (e.g., low, moderate, high, critical) as a function of test results.

The housing of device 200 and station 300 can be made of plastic, metal, composite, or any other material with structural and mechanical properties suitable for housing a lancet cartridge, test strip cartridge, electronics, and a linkage mechanism. Device 200 is preferably compact, with a height no more than 50 mm, a width no more than 17 mm, and a length no more than 100 mm. In some preferred embodiments, the housing of device 200 and station 300 comprises an outer protective shell made of molded plastic and an inner desiccant liner to minimize exposure to moisture.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

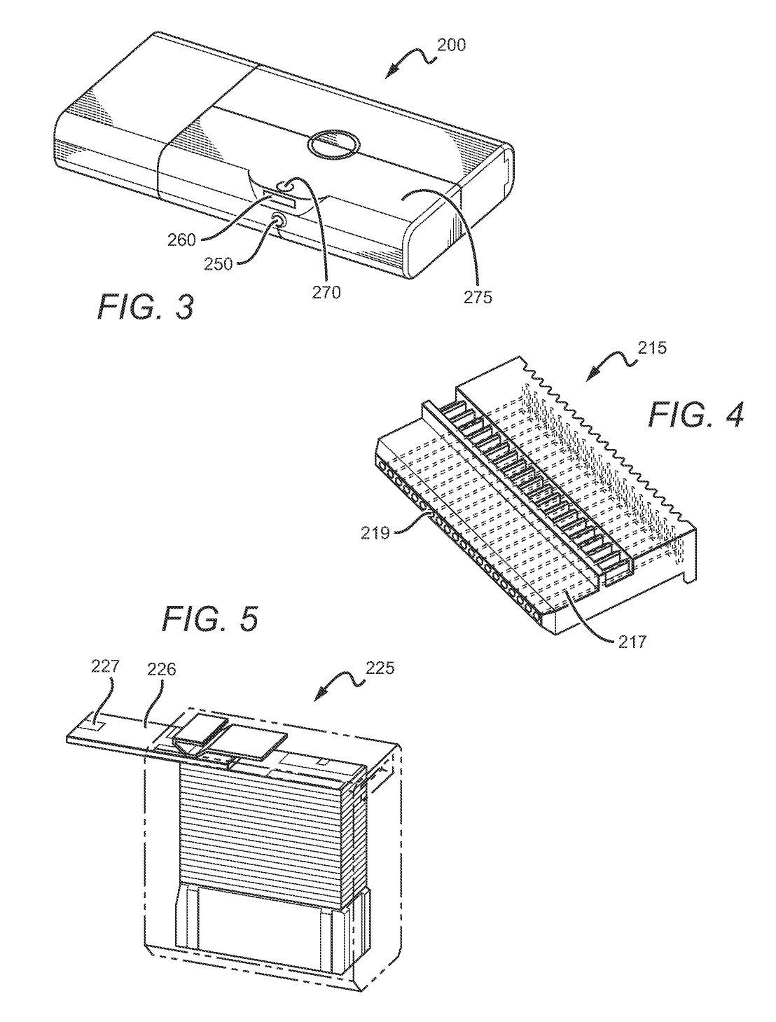

FIG. 3 shows the back side of device 200. Hole 250 is used to eject a lancet for pricking a body part in order to draw a blood sample. Wheel 260 is used to adjust the penetration depth of the lancet, while window 270 displays the penetration setting. Cover 275 is hingeably coupled to device 200 and can be opened in order to insert a lancet cartridge 215 and a analyte sensor cartridge 225 into device 200.

FIG. 4 shows one embodiment of a lancet cartridge 215. Cartridge 215 holds a plurality of lancets 217. Holes 219 are included on cartridge 215 to allow a lancet to be temporarily ejected from cartridge 215 and out of hole 250 of device 200 for drawing a blood sample. The lancet is safely retracted back into cartridge 215 by a retraction mechanism within device 200 (not shown).

FIG. 5 shows an analyte sensor cartridge 225. Cartridge 225 holds a plurality of test strips such as test strip 226. Strip 226 has an analyte sensor 227 for testing an analyte. Analyte sensors are well known and generally comprise an absorbent material with a reactant (e.g., analyte-binding reagent). Sensor 227 is configured to generate an electrical signal that is sent to the electronics of device 200 for conversion into readable test data. Acceptable test strip configurations that could be adapted for use within the existing subject matter is described in co-pending patent application having serial number XX/XXXXXX titled to Shaanan et al. “Test Unit Cartridge for Analyte Testing Device” filed on Jul. 20, 2011.

Cartridge 225 can include any appropriate number of test strips, preferably between 15 and 25, more preferably between 18 and 22, and most preferably 20. The number of test strips also preferably equals the number of lancets in cartridge 215, although other combinations are contemplated.

Cartridge 225 preferably includes analyte sensors configured to test for different analytes. For example, some sensors may test for glucose levels while other sensors test for fructosamine levels. Furthermore, cartridge 225 can have at least one test strip capable of testing for two analytes simultaneously, either by including two reactants within one absorbing material or by including two different analyte sensors on one test strip.

Cartridge 225 also preferably includes an inner desiccant liner for protecting the plurality of test strips from exposure to moisture. For example, a liner can be disposed between the test strips and the inner wall of cartridge 225, thus surrounding all the test strips (e.g., an inner sleeve). In addition, all cartridge apertures are preferably sealed with a pull-away adhesive label. Alternatively, a “sacrificial strip” can be included at the top of the stack of test strips. The sacrificial strip can be configured such that it corks and seals all apertures in cartridge 225. In this manner, cartridge 225 seals and protects the analyte sensors of the plurality strips from exposure to moisture and dust. The labels and/or sacrificial strip can be removed and discarded just prior to loading the cartridge into device 200. Cartridge 225 also preferably includes gaskets and/or o-rings at all cartridge apertures. These gaskets can be configured to mate with components of device 200 such that a seal is provided to protect the plurality of test strips from moisture while the cartridge is loaded in device 200 and not in use. It is also contemplated that lancet cartridge 215 could also include pull-away labels, seals, gaskets, and liners to protect the lancets from germs, bacteria, viruses, dirt, and other contamination.

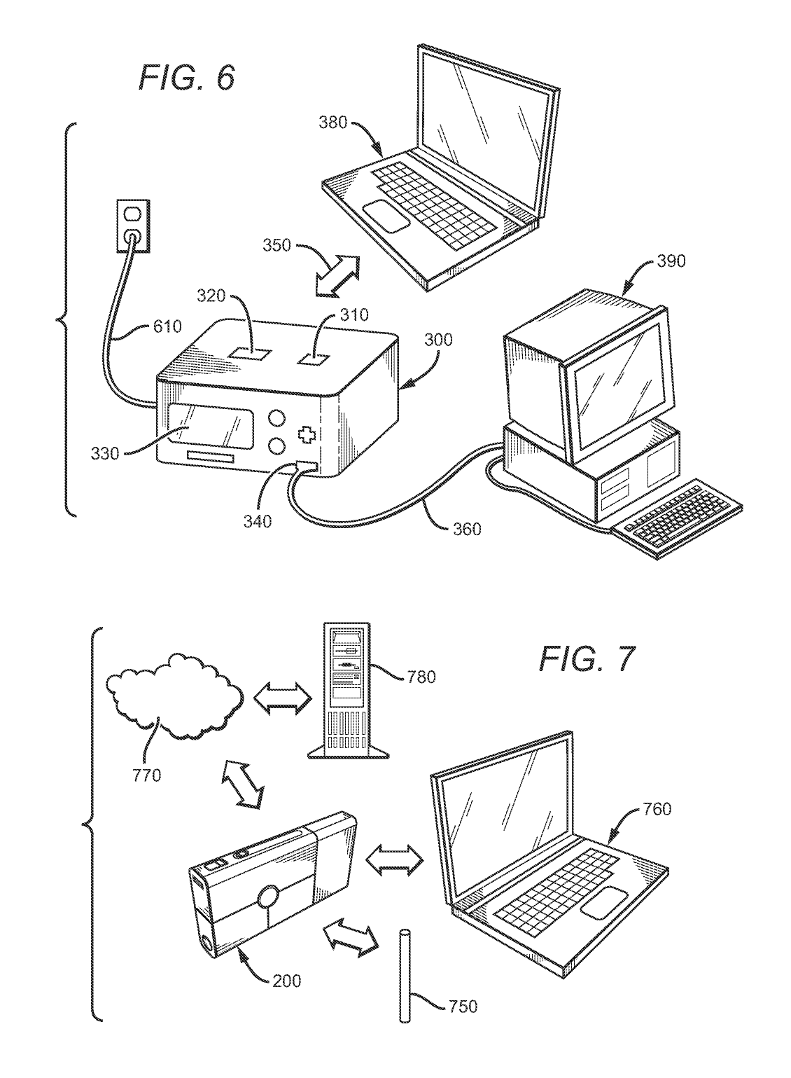

FIG. 6 shows docking station 300 communicatively coupled with laptop 380 and personal computer 390. Station 300 has a power cord 610 for supplying power to station 300 and device 200. Docking station 300 and laptop 380 are communicatively connected via wireless connection 350. Connection 350 can comprise any wireless protocol, for example Bluetooth, wifi, 802.11, and cellular networks. Personal computer 390 is connected to docking station 300 via wired connection 360 and data interface 340. In one embodiment, connection 360 is a USB cord and interface 340 is a USB port.

Connections 350 and 360 can be used to back up data, transmit data to a health care provider's server via the internet, reorder supplies, receive notifications from a doctor, or receive data analysis reports from analytics software running on the external device. Furthermore, it is contemplated that docking station 300 can connect to other external devices (e.g., smart phone, handheld health-monitoring device, insulin pen).

FIG. 7 shows testing device 200 in wireless communication with an insulin pen 750, laptop 760, and local area network (LAN) 770. In preferred embodiments, the insulin pen sends medication administration data (e.g., dosage administered, time of day, patient name) to device 200. The electronics of device 200 preferably correlates the medication administration data with the test data and non-test data, either time stamps or by some other correlative attribute. Device 200 can use communication with LAN 770 to upload data to a medical server 780, or otherwise exchange data with a third party. Alternative, LAN 770 could comprise a cellular network. It is also contemplated that device 200 could communicate with an external device (e.g., insulin pen, handheld health monitoring device) via a wired connection.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. An analyte testing system, comprising:
a hand-held analyte testing device capable of housing (i) a plurality of lancets, (ii) a plurality of analyte sensors usable in conjunction with the lancets, (iii) electronics for deriving test data from the analyte sensors, (iv) a visual display that displays the test data, and (v) a data recording facility that records non-test data;
a docking station having a power interface and a wired data interface that provide power and data connectivity to the device, respectively, and a storage facility that backs up both the test data and the non-test data; and
the hand-held analyte testing device further comprising electronics capable of backing up the test data and non-test data by wirelessly connecting to an external device other than the docking station.

2. The system of claim 1, wherein the docking station has a communication facility configured to transmit the test data and the non-test data to an external device.

3. The system of claim 1, wherein the electronics of the testing device has a communication facility configured to transmit the test data and the non-test data to an external device.

4. The system of claim 3, wherein the communication facility is configured to wirelessly transmit at least one of the test data and non-test data using any of at least two alternative wireless protocols.

5. The system of claim 3, wherein the communication facility is configured to communicate using a cell phone network.

6. The system of claim 3, wherein the external device comprises a medical server database.

7. The system of claim 3, wherein the external device comprises a network.

8. The system of claim 1, wherein the power interface supplies power to the testing device using an inductive pad.

9. The system of claim 1, wherein the non-test data comprises at least one item from the list consisting of text diary information, audio diary information, food eaten, minutes exercised, medication taken, and estimated calories burned.

10. The system of claim 1, wherein the non-test data comprises at least one item from the list consisting of supplies used, supplies ordered, inventory of lancets and test strips, and reorder history.

11. The system of claim 1, wherein at least one of the testing device and the docking station has a panic button.

12. The system of claim 1, wherein the docking station holds and charges a replacement battery for the testing device.

13. The system of claim 1, wherein the display comprises an LCD touch screen.

14. The system of claim 13, wherein the LCD touch screen displays a graph having a maximum threshold line, a minimum threshold line, and a test data line.

15. The system of claim 1, wherein the data recording facility includes a microphone.

16. The system of claim 1, wherein the data recording facility includes a keypad.

17. The system of claim 1, wherein at least one of the testing device and the docking station has a processor programmed to test adequacy of a communication link between the testing device and the docking station.

18. The system of claim 1, wherein at least one of the analyte sensors is configured to detect glucose.

19. The system of claim 1, wherein the docking station further comprises a processor and executable code that automatically backs up the test data and the non-test data.

20. The system of claim 1, wherein the docking station further comprises a processor and executable code that tracks supply usage, and automatically re-orders supplies.

21. The system of claim 1, wherein the plurality of lancets are contained in a lancet cartridge.

22. The system of claim 1, wherein the plurality of analyte sensors are contained in an analyte sensor cartridge.

23. The system of claim 1, wherein the plurality of lancets are contained in a lancet cartridge, and the plurality of analyte sensors are contained in a analyte sensor cartridge that is separate from the lancet cartridge.

24. The system of claim 1, wherein the storage facility is removable from the docking station.

25. The system of claim 1, wherein the storage facility is configured to store data in a generic file format.

26. The system of claim 25, wherein the format is selected from the group consisting of pdf, doc, xml, eps, html, jpeg, rtf, and txt.

27. The system of claim 1, wherein the storage facility is password protected and stores the test data and non-test data using encryption.

28. The system of claim 1, further comprising an insulin pen for administering a medication, wherein the pen includes a wireless communication facility configured to exchange data with the testing device.

29. the system of claim 13, wherein the docking station has a display screen.
